(19) United States
(12) Patent Application Publication
(10) Pub. No.: US 2026/0131410 A1
(43) Pub. Date: May 14, 2026

(54) Holding device, arrangement comprising the holding device, drug delivery device and method

(71) Applicant: Sanofi, PARIS (FR)

(72) Inventors: Tom Alexander Earwaker, Warwick Warwickshire (GB); Matthew Meredith Jones, Warwick Warwickshire (GB); David Aubrey Plumptre, Worcestershire (GB); Ian McFaul, Warwick Warwickshire (GB)

(21) Appl. No.: 19/129,595

(22) PCT Filed: Nov. 20, 2023
(86) PCT No.: PCT/EP2023/082329

§ 371 Date: May 13, 2025

(30) Foreign Application Priority Data
Nov. 21, 2022 (EP) 22315293.5

(51) Int. Cl. B23P 19/10; A61M 5/20

(52) U.S. Cl. CPC B23P 19/10; A61M 5/20; A61M 2205/8206; A61M 2207/10

(57) ABSTRACT

Disclosed is a holding device for holding an object during assembling of a medical device, comprising a holding structure and an alignment structure, wherein the holding structure comprises at least one opening, the alignment structure is configured to align an object relative to the holding structure, the object comprises a first side and a second side opposite to the first side of the object, the first side has a first surface configuration that is different to a second surface configuration of the second side, the at least one opening is arranged to apply a negative pressure to the first side of the object, and the holding device is configured to hold the object using the negative pressure only in a first orientation in which the first side of the object faces the at least one opening but not in a second orientation in which the second side faces the at least one opening.

### CROSS REFERENCE TO RELATED APPLICATIONS

The present application is the national stage entry of International Patent Application No. PCT/EP2023/082329, filed on Nov. 20, 2023, and claims priority to Application No. EP 22315293.5, filed on Nov. 21, 2022, the disclosures of which are incorporated herein by reference.

### BACKGROUND

The disclosure relates to a holding device that may be used to hold objects during assembling of a device. Pick and place technologies are widely used to assemble e.g. electronic devices. Usually, suctions heads are used that are positioned in the center of an object, e.g. an IC (integrated circuit), a discrete electronic component etc. Surface mounted devices (SMD) and/or IC packages, e.g. having a lead frame, may be assembled to a PCB (printed circuit board) or other circuit carrier by pick and place machines. Soldering of the PCB may be the next step in order to produce a complete PCBA (printed circuit board assembly) or other assembly. However, although the pick and place technology has been used for a long time, there may be limitations of the pick and place technology for special objects, e.g. for coin cells (button cells) and/or objects that have to observe an orientation. Costly image processing may be necessary in order to check or determine orientation of these objects during automatic pick and place.

### SUMMARY

The disclosure provides an improved holding device. The holding device is able to guarantee a right orientation of an object, e.g. during manual or automatic assembly. Furthermore, corresponding items and corresponding methods shall be provided.

According to an embodiment, a holding device for holding an object during assembling of a device, e.g. a medical device may comprise at least one, several or all of the following:
A holding structure, and
An alignment structure.

According to an embodiment, the holding structure may comprise at least one opening. The opening may be configured to provide a negative gas pressure, e.g. a suction gas flow that may come from the environment around the holding device and may be directed into the opening.

According to an embodiment, the alignment structure may be configured to align an object relative to the holding structure, e.g. laterally. This may allow positioning of the object relative to the at least one opening, especially positioning that allows dedicated holding of the object by the suction force applied through the at least one opening depending on the orientation of the object within the holding device.

According to an embodiment, the object may comprise a first side and a second side opposite to the first side of the object. The first side may have a first surface configuration that is different to a second surface configuration of the second side. This may allow dedicated holding of the object, e.g. by using the same gas flows interacting differently with the two sides. The object may be e.g. a battery, especially a non-rechargeable battery or a rechargeable battery. Another example of an object having two different surface configurations on opposite sides is a transistor within a plastic case, e.g. TO-92 (transistor outline) package. The right orientation of the device may be critical since otherwise destruction of the object and/or destruction of other component, especially of electronic components may be possible.

According to an embodiment, the at least one opening may be arranged to apply a negative pressure to the first side of the object. Providing negative pressure to the second side may be prevented or mitigated by the second surface configuration, e.g. by the “macro” profile, e.g. differences in the profile of at least 0.5 mm (millimeter) or of at least 1 mm. Alternatively or additionally, there may be differences in the “micro” profile of the surfaces, e.g. differences in the profile of less than 0.5 mm (millimeter).

According to an embodiment, the holding device may be configured to hold the object using the negative pressure only in a first orientation in which the first side of the object faces and/or may abut to the at least one opening but not in a second orientation in which the second side faces the at least one opening. Thus, dedicated holding is provided. Dedicated holding may be used to check the right orientation at least implicitly, e.g. the object is hold in the right orientation but not in the wrong orientation within the holding device.

According to an embodiment, the at least one opening may be at least one slit, e.g. at least one bended slit (arc shaped), at least one straight slit, or at least one hole, e.g. at least one round hole (circular), at least one an elliptical hole, etc. Combinations of different shapes of holes/slits may be used as well.

According to an embodiment, all openings may have the same shape. Alternatively, different shapes or shapes of different extensions may be used, e.g. in order to provide homogenous suction forces.

At least one abutting region may provide an abutment region or abutment surface for the object, e.g. in an axial direction. The at least one opening may be arranged in the abutting region. Alternatively, the at least one opening may be arranged at a separate position compared to the position of the at least one abutting region. The edge of the opening may be used as an abutting region, e.g. the whole edge or only a part of the edge.

According to an embodiment, the device may be a medical device, e.g. a drug delivery device, comprising electronic components or an electronic module for generation and/or storage of data, e.g. of digital data related to drug delivery. If a coin cell is applied to a medical device, guaranteeing the right orientation (e.g. polarity) of a connection to an electronic circuit may be important to lives of patients. The medical device may be a drug delivery device, e.g. an auto-injector, an electronic thermometer, a pace maker, etc. However, the right polarity may be also important in other devices, e.g. key fobs, especially for locking systems, remote control device, etc.

According to an embodiment, the negative pressure may be applied through a pneumatic system, e.g. a system comprising a control unit and/or a system that may be controlled by an operator. Thus, it may be possible to apply the suction force or to stop the suction force, e.g. by usage of a foot switch or other appropriate switch. However, automatic release of the object from the holding device may be used as well. Switching off of negative pressure may release the object in a simple manner from the holding device. However, additionally or alternatively, other release method(s) may be used, e.g. using a tool to remove the object from the holding device.

The object may be manually inserted into holding device. Insertion in an incorrect orientation may e.g. prevent further transport of the object. However, automatic insertion is possible as well.

One technical effect of the holding device may be that it enables to selectively hold and/or to selectively transport the object in the right orientation against the force of gravity. In the wrong orientation, a holding force may not be strong enough and the object may fall out of holding device by gravity. Thus, an object orientation selective holding device (ooshd) may be provided. Different surface configurations may be realized due to different geometrical shapes, e.g. within an edge region, and/or different surface structure and/or different size and/or different diameter in case of a circular first surface and a circular second surface. This will be described in more detail below. A first surface of the object and a second surface of the object may face in opposite directions.

The holding device may be configured to hold the object in the holding device when the first surface faces the holding structure and may not hold the object in the holding device when the second surface faces the holding structure.

Image processing may not be necessary anymore to detect the orientation of the object or may be used additionally, e.g. as a double check. Thus, a very simple and/or cost efficient solution for orientating the object is provided by the holding device. Errors of a human operator who assembles a device, e.g. inserts the object may be prevented. However, there may be also errors in automatic production that may be prevented using the proposed holding device.

Usage of gas/air may be cost effective, e.g. compared to other complicated mechanically operated holding devices and/or to image processing, etc.

According to an embodiment, the alignment structure may comprise at least one alignment region arranged to align the object laterally. The at least one alignment region may be arranged adjacent to the at least one opening, preferably in order to hold the object at the at least one edge region of the object and/or to provide high accuracy of lateral alignment. “Adjacent” may mean in the range of 0.5 percent to 5 percent of maximum lateral extension of object. At least one inlet opening may be positioned preferably near or adjacent to a lateral alignment surface of the alignment structure. A distance between a lateral alignment surface and the opening (e.g. edge of the opening) may be less than 2 mm (millimeter) or less than 1 mm, preferably more than 0.5 mm to give an example for a lower limit.

The border (boundary) or edge region of the object may be especially appropriate to realize dedicated holding for objects having different edge regions on different sides, e.g. coin cells, transistors in plastic housings, etc. Thus, the object may be hold in the edge region in the right orientation but not or only slightly at a center region, e.g. because in the center region, there is not a different surface configuration which would allow selective or dedicated holding of the object within the holding device.

According to an embodiment, the holding structure may comprise at least one abutting region configured to abut the second side of the object or alternatively the first side and the second side of the object.

According to a first embodiment, the at least one opening may be arranged at a different position compared to the at least one abutting region. According to the first embodiment, the at least one opening may only be covered if the object is arranged in the holding device in the first orientation but not if the object is arranged in the holding device in the second configuration.

Alternatively or additionally, the at least one opening may be arranged in the at least one abutting region. According to the second embodiment, the at least one opening may be covered if the object is arranged in the holding device in the first orientation and also if the object is arranged in the holding device in the second configuration. In this case e.g. different surface roughness may be used to reach selective holding of the object only in the first orientation.

According to an embodiment, the different surface configuration may be realized due to at least

one of the following features:
a) A greater maximal lateral extension or diameter of a most prominent first surface on the first side of the object compared to the maximal lateral extension or diameter of a most prominent second surface on the second side of the object. Preferably, the first surface may have a greater surface area compared to the second surface.
b) A smaller surface roughness of a first surface on the first side of the object compared to the surface roughness on a second surface on the second side of the object. Preferably, the first surface may have a smoother surface profile compared to the second surface.
c) A chamfered or rounded circumferential region on the first side of the object, wherein no such chamfered or rounded circumferential region is present on the second side of the object. The second side may have a comparably sharp edge, e.g. compared to an edge on the chamfered or rounded circumferential region. The chamfer may extend preferably along the whole circumference. The rounding may not only be present in a circumferential direction but also in a cross section that comprises the longitudinal axis of the object and preferably also a radial axis. Thus rotation of the object does not matter and rotational alignment may not be necessary. However, the chamfer may also extend only in at least one region of the circumference of the object but not in at least one other circumferential region of the object. Thus, e.g. two chamfered or rounded circumferential region may be present on the first side of the object, preferably at opposite lateral positions.
d) Other physical characteristics appropriate to interact with a gas (e.g. air) stream through the at least one opening in order to achieve dedicated holding of the object depending on its orientation relative to the holding device.

According to an embodiment, the holding device may comprise at least one, an arbitrarily selected or all of the following features:
a) wherein an abutting region is arranged to provide an abutment surface for the object in a first direction, preferably in an axial direction of the object. The alignment structure may comprise at least one lateral holding surface or surface portion that is configured to align the object in a second direction that is different from the first direction,
b) wherein the holding device comprises a gas transport structure. The gas transport structure may comprise at least one channel or tube connected to the at least one opening or connectable to the at least one opening and configured to be connected to a negative pressure supply source.

According to an embodiment, the holding device may comprise a housing or casing. The holding structure and/or the alignment structure may be provided on the housing or within the housing. The housing may comprise at least a part of a gas transport structure. “Provided” may mean that the respective structure may be an integral part of the housing or may be arranged on the housing, e.g. as a separate part. The gas transport structure is described below for other embodiments. Usage of a housing may allow to realize a compact holding device.

According to a further embodiment the housing may comprise at least one, several or all of the following:
A cylindrical outer shell surface. A rotation symmetry axis of the cylindrical outer shell surface may define a longitudinal axis of the housing,
A circular proximal surface, and
An annular or essentially annular distal surface, e.g. not regarding air flow (leakage) recesses as described in this document.

The housing may comprise a cylindrical retaining space extending e.g. from the annular or essentially annular distal surface along the direction of the longitudinal axis of the housing up to a circular abutting surface. The circular abutting surface may be preferably arranged coaxially to the longitudinal axis of the housing. The cylindrical retaining space may be part of the alignment structure. The circular abutting surface may comprise the at least one opening of the pneumatic subsystem, preferably in a circumferential region. The circular abutting surface may be a plane surface.

According to a further embodiment, the housing may comprise preferably at least one lateral main channel extending radially and connected to the at least one opening. The at least one main channel may be configured to be connected with at least one tube or hose, e.g. by inserting the tube or hose into an open end of the at least one main channel.

The cylindrical outer shell surface may extend from the circular proximal surface to the annular distal surface.

Thus, the housing may be adapted to the assembly of the object, e.g. an essentially flat cylindrical shaped object, into a medical injection device, especially a pen-device or auto-injector having a pen-shape. Exemplary, the cylindrical outer surface may allow easy alignment of the holding device with a cylindrical housing of drug delivery device or with a cylindrical housing of an electronic module that is configured to be to be connected with drug delivery device, e.g. mechanically. Other technical effects mentioned above may apply to the housing as well.

According to an embodiment, the holding device may comprise a cylindrical retaining space that may be adapted to retain the object and that is delimited by the holding structure and/or by the alignment structure. The diameter of the cylindrical retaining space may be slightly greater compared to the maximal diameter of the object, e.g. at most one percent greater or at most two percent greater but at least e.g. 0.5 percent of diameter of object greater to give an example for a lower limit. The height of the cylindrical retaining space may be preferably within the range of one half of maximal height of the object to the maximal height of the object. Thus, insertion of the object into the holding device may be easy. Moreover, release of the object may be possible by switching of the negative pressure and using gravity only. However, it may also be possible to promote release by providing a positive pressure through the at least one opening and/or by using a separate tool. The cylindrical retaining space may be most appropriate for cylindrical coin cell batteries since the cylindrical retaining space may be complementary to the cylindrical outer shape of the object. Easy alignment, especially in all lateral directions may be possible using a cylindrical retaining space. However, usage of separate ribs at the border of a cylindrical retaining space or using ribs without usage of a cylindrical retaining space at all is also feasible, e.g. three ribs or more than three ribs.

According to an embodiment, at least one first opening of the at least one opening may be arranged on a first location. At least a one second opening of the at least one opening may be arranged on a second location. The first location and the second location may be arranged on opposite lateral sides of the holding structure. The first location and the second location may correspond to opposite peripheral regions of the first side of the object.

Thus, the holding device may be configured such that dedicated holding of the object is realized depending on the obstruction (e.g. closing or almost closing) of the first opening by a first peripheral region of the first side and of the at least second opening by a second peripheral region of the first side. The first peripheral region may not obstruct or close the second opening thereby. The second peripheral region may not obstruct or close the first opening thereby. “Peripheral region” may relate to a portion that is an outmost portion and that is in the range of e.g. 60 percent to 100 percent or in the range of 75 percent to 100 percent of the radius of the first side of the coin cell battery or other object and/or of an receiving space of the coin cell or of another object within the holding device.

Preferably, the at least one first opening may be part of a first group of the at least one opening. The at least one second opening may be part of a second group of the at least one opening. This may enable simple holding of the object within the holding device on opposite lateral sides (e.g. disc shaped object) or ends (e.g. bar shaped object). A greater holding area may be provided if a group of openings is used. This may result in more secure holding and/or in redundancy, e.g. if one of the openings or holes is closed by dust, etc.

According to a further embodiment the holes may be arranged around the whole circumference of a retaining space, e.g. in order to provide a larger holding force and/or a more homogeneous holding force.

According to an embodiment, a gas transport structure of the holding device may comprise at least one of the following:
a) At least one main channel that may be connected with at least a part of the at least one opening. The main channel may have a larger cross section area, e.g. in a cross section that includes an angle with the main flow direction in the range of 80 degrees to 100 degrees, compared to the cross section of minor channels which lead to the main channel. Thus, the main channel may bundle the flow of several minor channels.
b) A first main channel that may be fluidically connected with at least a part of the openings of the at least one opening of a first group and a second main channel that may be different from the first main channel and that may be fluidically connected with openings of a second group. Usage of two main channels may make the gas transport structure simple, e.g. avoiding complicated inner channel(s). Alternatively, more than two main channels or more than three main channels may be used.
c) At least one gas flow (leakage) recess adjacent (e.g. near) to the at least one opening, e.g. a laterally (or radially) outwardly recessed recess and/or an axially recessed recess. The at least one gas flow (e.g. gas leakage) recess may be arranged such that the gas flow between the at least one gas flow recess and the at least one opening may be restricted essentially (blocked) by the object when the first side of the object faces the at least one opening, resulting in a comparably high suction force of the object to the holding device, and may be enabled (essentially unblocked) when the second side of the object faces the at least one abutting region and/or at least one opening, resulting in a low suction force for holding the object. Thus, the object may form a wall, e.g. a wall portion, of a gas channel formed by the at least one gas flow recess and the at least one opening when the second side of the object faces the at least one opening. At least two functions may be combined by providing the at least one gas flow (leakage) recess, e.g. providing an air flow channel and providing lateral alignment.

According to an embodiment, the at least one gas flow recess may be configured such that at least two gas channels are formed between the at least one gas flow recess and at least two of the at least one opening when the second side faces the at least one opening. The at least two gas channels between the at least one gas flow recess and the at least two openings may be blocked or may be at least essentially blocked by the object when the first side faces the at least one opening. Providing a gas flow (leakage) recess for several ones of the openings may simplify the holding device. However, 1:1 arrangements of gas flow recesses and openings of the at least one opening may be used as well.

According to a further embodiment, the at least one gas flow (leakage) recess may be a cylindrical recess or an almost cylindrical recess, e.g. open only on one lateral side of the cylinder, especially adjacent to the retaining space. Cylindrical recesses may be easily produced using a drill tool. However, other shapes may be used as well.

According to an embodiment, the holding device may comprise at least one main channel that may be fluidically connected to at least one of the openings and that may redirect the gas flow from a first flow direction in a suction channel beginning e.g. at the at least one opening to a second flow direction, wherein an angle between the first direction and the second direction may be in the range of 80 degrees to 100 degrees, preferably about 90 degrees or 90 degrees.

Preferably, the main channel may comprise at least one, several (an arbitrary selection of) or all of the following:
a) An inlet portion comprising at least one inlet opening fluidically connected to a suction channel coming from the at least one opening.
b) A tube offset structure or hose offset structure arranged downstream to an inlet portion of the main channel and configured to define an offset between a sidewall of the main channel and a tube/hose fluidically connected to the main channel, preferably on the end of the tube/hose arranged within the main channel.
c) An outlet portion comprising at least one or only one outlet opening, preferable a cylindrical outlet portion or an almost cylindrical outlet portion.

The inlet portion may have a cylindrical lower wall. The cylindrical wall, e.g. about half of a cylinder may laterally be bordered by a flat circular surface or flat surface of semicircular shape at one side. The inlet portion may be open on a side that is opposite to the flat surface. The open side may lead to other parts of the gas transport structure, e.g. a space configured to retain one end of a tube or hose.

Thus, the inlet openings on the cylindrical lower wall may have different lateral offsets to the flat surface due to circular arrangement of at least one opening to hold object and to usage of straight channels from these openings to the inlet portion, e.g. of straight suction channels that are arranged parallel or essentially parallel to the flat surface.

Alternatively, the inlet portion may be a curved portion, following the curvature of the circular arc arrangement of the at least one opening to hold the object. Again, there may be a lower cylindrical wall that comprises portions of a cylindrical surface. The cylindrical wall may be bordered by a curved circular or semicircular surface. Same distances of inlet openings to the curved surface may be used. Similar as described above, the inlet portion may be open on a side that is opposite to curved surface. There may be straight suction channels between the at least one respective opening and a respective one inlet opening. The straight suction channels may be aligned along a curve following the curve along which the at least one opening is arranged, e.g. circular arc shaped curves.

The tube offset structure or hose offset structure may provide a holding function and/or a sealing function for the tube/pipe or for a flexible hose. Usage of flexible hoses may make movements of the holding device easier, e.g. by manual operation. The tube offset structure or hose offset structure may be arc shaped, e.g. circular arc shaped, or may have the shape of a half disc, or any other appropriate shape.

The offset structure may preferably extend only around a portion of the circumference of a circular side face, e.g. in the range of more than one quarter to less than three quarter of the circumference. The other portion(s) of the circumference may be used as an air channel forming a connection between channel(s) to the at least one opening and other parts of the gas transport structure. Thus, the offset structure may form a stop face for the tube or hose arranged on it.

However, alternatively an external flange may be used to connect a tube/pipe/hose to the main channel by pulling or pushing the pipe or tube over the flange. This may provide better fit than internal arrangement of tube or pipe, e.g. less air leakage, etc.

The outlet portion of the main channel may be configured to form an inner surface that may interact with an outer surface of a tube/pipe or hose to form an air tight connection without further sealing means or using further sealing means, e.g. gasket, sealing rings, etc.

The main channel may be optimized fluid-dynamically to generate a homogenous flow profile at the openings within the retaining space.

According to a further embodiment, the holding device may be manufactured by 3D (three dimensional) printing. A layer by layer technique may be used (additive manufacturing), e.g. contrary to molding techniques using an expensive mold and an expensive injection molding machine. Thus, cost effective production may be used, even if only a low number of holding devices is produced, e.g. less than 1000, but e.g. more than 10 or even more than 100. Filament printing or lithographic printing may be used. Optionally a support material may be used, e.g. solvable in water or other chemical solution selectively to a main material of the holding device. Furthermore the CAD (computer aided design) data of the 3D printing model may be protected.

Alternatively, injection molding may be used to produce a holding device based on plastic material. However, a metal may also be used as material for the holding device, e.g. using cutting production machines (subtractive manufacturing).

According to a further aspect, a holding system may comprise:
A holding device according to any one of the preceding embodiments. The holding device may comprise at least one opening that is positioned in a region in which the at least one opening is covered by an object in its correct orientation and not covered or less covered by the object in its incorrect orientation.
A negative pressure source, e.g. a pump, and
A connection system connecting or configured to connect the holding device and the negative pressure source. The above mentioned gas transport structure may be part of the connection system. The connection system may be part of a pneumatic system. The pneumatic system may comprise e.g. a control unit and/or at least one valve. The control unit may control the negative pressure source and/or the valve to stop or interrupt delivery of the negative pressure, e.g. in order to release object from holding device.

Thus, the technical effects mentioned above for the holding device may also apply to the holding system.

According to an aspect, the above mentioned object is solved by an arrangement. The arrangement may comprise:
The holding device according to any one of the embodiments mentioned above or the holding system according the embodiment mentioned above, and
An object configured to be held in the holding system in order to place the object into a device during assembling of the device.

Thus, the technical effects mentioned above for the holding device may also apply to the arrangement.

According to an aspect, a method of assembling a drug delivery device or an electronic module for a drug delivery device is provided. The method may comprise at least one, an arbitrary selection of or all of the following:
Providing or using a holding device according to any one of the embodiments mentioned above or providing the holding system according to the embodiment mentioned above.
Placing the object in the holding device. The object may be held within the holding device when the first side faces the at least one opening, e.g. right orientation. The object or another object, e.g. of the same type, especially having the same outer shape may fall out of the holding device when the second side faces the at least one opening, e.g. wrong orientation. This may be regarded as an implicit check whether the object is placed in the right or wrong orientation within the holding device.
Placing the object into or onto an object receiving space within e.g. the drug delivery device or within e.g. the electronic module using the holding device. There may be a relative movement between the holding device and the device or module to be assembled.

Thus, it is make sure that the object is placed with the right orientation during assembling of the device or module, e.g. with the right electrical contact(s) to the plus contact and to the minus contact of an electrical circuit. It may be make sure, that the electrical circuit will operate properly. Importantly, especially in the medical field, e.g. lives of patients may depend on the right orientation and/or on the right polarity of assembling an object, e.g. a coin cell into the device.

If the object is placed into the holding device with the wrong orientation, due to gravity and due to a low holding force it will fall out of the holding device. If it is attempted to transport the object with the wrong orientation to a desired location, this will not be possible.

Release of the object may be realized by interrupting the negative pressure, e.g. switching off a pump or cut off the air flow in an appropriate manner, e.g. using a valve. A pump of a pneumatic subsystem may be switched off in order to release e.g. an electric battery (e.g. coin cell) from the holding device. Alternatively and or additionally, a tool may be used to release e.g. the electric battery from the holding device, e.g. inserting the tool into the gas leakage recesses that are mentioned above or pushing from above through a hole extending through the holding device up to the retaining space.

Thus, the technical effects mentioned above for the holding device may apply as well to the method and vice versa.

According to an aspect, a drug delivery device is provided, especially a drug delivery device manufactured or manufacturable (producible) using the method mentioned above. The drug delivery device may be a pen-type device, e.g. of the axial extension type. The drug delivery device may comprise at least one, several arbitrarily selected or all of the following:
Preferably a housing, e.g. a housing that comprises a drive mechanism.
A container receptacle, e.g. provided on the housing or within the housing. The container receptacle may be configured to receive a container comprising a drug, see e.g. the drug list mentioned below. Preferably the container containing a drug may be also comprised within the drug delivery device, e.g. the container may be arranged in the container receptacle. The container may be a cartridge comprising a coupling structure that is coupled or that may be coupled to a needle, e.g. to a needle having two sharp ends.
An electronic module, preferably arranged within or on the housing. The electronic module may be configured to detect drug delivery, especially the amount of a delivered or selected dose of the drug. The electronic module may be electrically powered by an electrical power source comprised within or formed by the object. A first surface of the object may be arranged more proximally compared to a second surface of the object. The first surface may be a flat surface that may have a first maximal lateral extension or diameter. The second surface may be a flat surface that may have a second maximal lateral extension or diameter. Preferably, the first maximal lateral extension or diameter may be greater than the second maximal lateral extension or diameter, preferably by at least 1 percent or at least 2 percent of the second maximal lateral extension or diameter. Preferably, the first surface may have an opposite electrical polarity compared to the second surface. The first maximal lateral extension or diameter may not be greater than 10 percent or 5 percent of the second maximal lateral extension or diameter.

Thus, the technical effects mentioned above for the holding device may apply as well to the drug delivery device assembled using the holding device.

The manufacturing of the drug delivery device may enable usage of standardized coin cells, e.g. the plus electrode may have a greater surface compared to the surface of a minus electrode. A chamfer or rounding may be present on the side of the coin cell on which the negative electrode is arranged.

Distal may refer to an end of the drug delivery device that is near to the patient during usage of the drug delivery device, e.g. to the needle end. Proximal may refer to the opposite side, e.g. to a needle far end.

The drug delivery device may comprise at least one, several arbitrarily selected or all of:
A plunger configured to interact with a stopper in a syringe or in another medicament container, e.g. in a cartridge.
A manually driven drive sleeve, e.g. extending proximally during dose setting.
A retaining space for a container comprising a drug.
Optionally, the container itself,
A dose setting mechanism, e.g. comprising a number sleeve.
A cap, etc.

The drug may be a drug as mentioned below in the list of drugs, e.g. insulin.

The electronic module may be configured to detect drug delivery and/or to detect or measure the amount of drug delivered and/or set. The electronic module may comprise a memory configured to store data about the drug, drug delivery time, and/or drug delivery amount. Moreover, the electronic module may be configured to establish a data communication link to another device, e.g. a smartphone or a computer device. Thus, the electronic module may be configured to send data and/or to receive data, e.g. digital data. Moreover, the electronic module may comprise any of the parts that are mentioned in the following.

According to a further aspect, an electronic module for a drug delivery device is provided, especially an electronic module manufactured using a holding device according to any one of the preceding embodiments. The electronic module may be configured to be mechanically connected to a drug delivery device that is configured to deliver a drug, especially to a proximal end of the drug delivery device. The electronic module may be configured to detect drug delivery, especially the amount of a delivered or selected (set) dose of drug. The electronic module may be powered by an object or by the object, e.g. by a coin cell. A plus electrode of the object may be arranged more proximally compared to the minus electrode of the object, e.g. a coin cell. The meaning of “proximal” may be the same as mentioned above and used for the electronic module coupled to the drug delivery device.

Moreover, the electronic module may be configured to establish a data communication link to another device, e.g. a smartphone or a computer device. Bluetooth (SIG, Special Interest Group), Bluetooth low energy or other appropriate data communication protocol may be used, e.g. ZigBee (ZigBee-Alliance), etc. The electronic module may comprise:
A microprocessor or a microcontroller (comprising more peripheral circuits compared to a microprocessor that may be configured to execute operation code stored in a memory. Alternatively, another control unit may be used, e.g. a finite state machine (ASIC (application specific circuit), etc.)
At least one motion detection sensor, e.g. rotation or translation.
At least one switch for switching to a second one of two operation modes, e.g. to a high power mode that may consume much more power compared to a low power mode. The low power mode may be e.g. selected by a further switch or by the lapse of a pre-given time without user actions.

According to a first embodiment, the sensor may comprise e.g. at least one light pipe or two light pipes, e.g. each light pipe sending radiation to a rotating part, e.g. having teeth and recesses between adjacent teeth, and each light pipe receiving reflected light from the rotating part. This may be an axial detection. This embodiment may allow combining the electronic module with drug delivery devices that may be used also without an electronic module. An angular offset may be less than 30 degrees id e.g. two light pipes are used.

According to a second embodiment, the sensor may comprise e.g. at least one or at least two pairs of radiation source and radiation detector, e.g. LED (light emitting diode) and photodiode or phototransistor, e.g. arranged around the circumference of an encoder ring or other rotating part, e.g. at angle of about 135 degrees or other appropriate value, e.g. within a range of 10 degrees to 140 degrees. The sensors may be arranged on a flexible PCB, e.g. in order to enable bending of the sensors into its final sensing position and/or avoiding separate electronic connections between the sensors and a main control unit of the electronic module, e.g. processor, etc.

According to a further aspect, a method of holding an object is provided, especially during assembling of a drug delivery device or of an electronic module for a drug delivery device, comprising at least one, an arbitrarily selection of or all of the following:
Laterally aligning an object. The object may comprise a first side and a second side that is opposite to the first side of the object. The first side may have a different surface configuration compared to the second side.
Applying a negative pressure through at least one opening to the aligned object, e.g. aligned using e.g. an alignment structure.
Realizing dedicated holding of the object depending on the side of the object that is adjacent to the at least one opening and depending on the obstruction of the at least one opening by the respective one of the sides of the object, e.g. by an obstruction by an edge region of the object, preferably at opposite lateral sides of the first side of the object.

Thus, the technical effects mentioned above for the holding device may apply as well to the method and vice versa.

According to a further embodiment the method may comprise holding the object in a lateral region thereof but preferably not in a central region. Details are mentioned above, see description of the arrangement of the at least one opening. Holding the object laterally may provide better possibilities for dedicated holding depending on the orientation of the object due to different surface configurations on both relevant sides of the object. The method may be combined with any of the embodiments mentioned above.

The present application has the priority of application EP 22315293.5 filed on November 2022 at the EPO, the disclosure of which is herewith explicitly incorporated by reference into the present disclosure for all legal purposes.

In the following, a set of aspects is disclosed. The aspects are numbered to facilitate referencing the features of one aspect in other aspects. The aspects form part of the disclosure of the present application and could be made subject to independent and/or dependent claims irrespective of what currently is claimed in the application and also independent of the references in brackets.
1. Holding device (10) for holding an object (O) during assembling of a medical device, comprising:

a holding structure (HS), and

an alignment structure (AS),

wherein the holding structure (HS) comprises at least one opening (21-1 to 21-4; 22-1 to 22-4),

wherein the alignment structure (AS) is configured to align an object (O) relative to the holding structure (HS),

wherein the object (O) comprises a first side (62) and a second side (66) opposite to the first side (62) of the object (O),

wherein the first side (62) has a first surface configuration that is different to a second surface configuration of the second side (66),

wherein the at least one opening (21-1 to 21-4; 22-1 to 22-4) is arranged to apply a negative pressure to the first side of the object (O), and

wherein the holding device (10) is configured to hold the object (O) using the negative pressure only in a first orientation in which the first side (62) of the object (O) faces the at least one opening (21-1 to 21-4; 22-1 to 22-4) but not in a second orientation in which the second side (66) faces the at least one opening (21-1 to 21-4; 22-1 to 22-4).
2. Holding device (10) according to aspect 1, wherein the alignment structure (AS) comprises at least one alignment region provided to align the object (O) laterally, and

wherein the at least one alignment region is provided adjacent to the at least one opening (21-1 to 21-4; 22-1 to 22-4), preferably in order to hold the object (O) at at least one edge region of the object (O).
3. Holding device (10) according to aspect 1 or 2, wherein the holding structure (HS) comprises at least one abutting region (19) configured to abut the second side of the object or alternatively the first side (62) and the second side (66),

wherein the at least one opening (21-1 to 21-4; 22-1 to 22-4) is arranged at a different position compared to the at least one abutting region (19) or wherein the at least one opening (21-1 to 21-4; 22-1 to 22-4) is arranged in the at least one abutting region (19).
4. Holding device (10) according to any one of the preceding aspects, wherein the different surface configuration may be due to at least one of the following features:
a) a greater maximal lateral extension or diameter of a most prominent first surface (64) on the first side (62) compared to the maximal lateral extension or diameter of a most prominent second surface (68) on the second side (66),

wherein preferably the first surface (64) has a greater surface area compared to the second surface (68),
b) a smaller surface roughness of a first surface (64) on the first side (62) compared to the surface roughness on a second surface (68) on the second side (66),
c) a chamfered or rounded circumferential region on the first side (62), wherein no such circumferential region is present on the second side (66),
d) other physical characteristics appropriate to interact with a gas stream through the at least one opening (21-1 to 21-4; 22-1 to 22-4) in order to achieve dedicated holding of the object (O) depending on its orientation relative to the holding device (10).
5. Holding device (10) according to any one of the preceding aspects, comprising at least one of the following features:
a) wherein at least one abutting region (19) is arranged to provide an abutment surface for the object (O) in a first direction, preferably in an axial direction of the object (O), and

wherein the alignment structure (AS) comprises at least one lateral holding surface (20) or surface portion that is configured to align the object (O) in a second direction that is different from the first direction,
b) wherein the holding device (10) comprises a gas transport structure (GTS),

wherein the gas transport structure (GTS) comprises at least one channel (23-1 to 23-4; 24-1 to 24-4; 25-1, 25-2) or tube (40-1, 40,2) connected to the at least one opening (21-1 to 21-4; 22-1 to 22-4) or connectable to the at least one opening (21-1 to 21-4; 22-1 to 22-4) and configured to be connected to a negative pressure supply source.
6. Holding device (10) according to any one of the preceding aspects, comprising a housing (H),

wherein the holding structure (HS) and/or the alignment structure (AS) is provided on the housing (H) or within the housing (H), and

wherein the housing (H) comprises at least a part of a gas transport structure (GTS).
7. Holding device (10) according to any one of the preceding aspects, comprising a cylindrical retaining space (18) that is adapted to retain the object (O) and that is delimited by the holding structure (HS) and/or by the alignment structure (AS).
8. Holding device (10) according to any one of the preceding aspects, wherein at least one first opening (21-1 to 21-4) of the at least one opening (21-1 to 21-4; 22-1 to 22-4) is arranged on a first location and wherein at least a one second opening (22-1 to 22-4) of the at least one opening (21-1 to 21-4; 22-1 to 22-4) is arranged on a second location,

and wherein the first location and the second location are arranged on opposite lateral sides of the holding structure (HS),

wherein preferably the at least one first opening (21-1 to 21-4) is part of a first group (G1) of the at least one opening (21-1 to 21-4; 22-1 to 22-4) and/or wherein the at least one second opening (22-1 to 22-4) is part of a second group (G2) of the at least one opening (21-1 to 21-4; 22-1 to 22-4).
9. Holding device (10) according to any one of the preceding aspect, especially according to aspect 5, wherein a gas transport structure (GTS) comprises at least one of the following:
a) at least one main channel (25-1, 25-2) that is connected with at least a part of the at least one opening (21-1 to 21-4; 22-1 to 22-4),
b) a first main channel (25-1) that is fluidically connected with at least a part of the at least one opening (21-1 to 21-4) of a first group (G1) and a second main channel (25-2) that is different from the first main channel (25-1) and that is fluidically connected with at least a part of the at least one opening (22-1 to 22-4) of a second group (G2),
c) at least one gas flow recess (29-1 to 29-3; 29-4 to 29-6) adjacent to the at least one opening (21-1 to 21-4; 22-1 to 22-4), wherein the at least one gas flow recess (29-1 to 29-3; 29-4 to 29-6) is arranged such that the gas flow between the at least one gas flow recess (29-1 to 29-3; 29-4 to 29-6) and the at least one opening (21-1 to 21-4; 22-1 to 22-4) is restricted essentially by the object (O) when the first side of the object (O) faces the at least one opening (21-1 to 21-4; 22-1 to 22-4) and is enabled when the second side of the object (O) faces at least one of the at least one opening (21-1 to 21-4; 22-1 to 22-4) and the at least one holding region (19).
10. Holding device (10) according to aspect 9, wherein the at least one gas flow recess (29-1 to 29-3; 29-4 to 29-6) is configured such that at least two gas channels are formed between the at least one gas flow recess (29-1 to 29-3; 29-4 to 29-6) and at least two of the at least one opening (21-1 to 21-4; 22-1 to 22-4) when the second side (66) faces the at least one opening (21-1 to 21-4; 22-1 to 22-4), and

wherein the at least two gas channels between the at least one gas flow recess (29-1 to 29-3; 29-4 to 29-6) and the at least two openings (21-1 to 21-4; 22-1 to 22-4) are blocked or at least essentially blocked by the object (O) when the first side (62) faces the at least one opening (21-1 to 21-4; 22-1 to 22-4).
11. Holding device (10) according to any one of the preceding aspects, comprising at least one main channel (25-1, 25-2) that is fluidically connected to at least one of the openings (21-1 to 21-4; 22-1 to 22-4) and that redirects the gas flow from a first flow direction at the at least one opening (21-1 to 21-4; 22-1 to 22-4) to a second direction, wherein an angle between the first direction and the second direction is in the range of 80 degrees to 100 degrees, preferably about 90 degrees or 90 degrees,

and wherein preferably the main channel (25-1, 25-2) comprises at least one of the following:
a) an inlet portion comprising at least one inlet opening fluidically connected to a suction channel coming from the at least one opening (21-1 to 21-4; 22-1 to 22-4),
b) a tube or hose offset structure (27) arranged downstream to an inlet portion of the main channel and configured to define an offset between a sidewall of the main channel and a tube fluidically connected to the main channel,
c) an outlet portion comprising an outlet opening, preferably an essentially cylindrical outlet portion or a cylindrical outlet portion.
12. A holding system comprising:

a holding device (10) according to any one of the preceding aspects,

a negative pressure source, and

a connections system connecting or configured to connect the holding device (10) and the negative pressure source.
13. Arrangement (1) comprising the holding device (10) according to any one of the aspects 1 to 11 or the holding system according to aspect 12 and an object (O) configured to be held in the holding system in order to place the object (O) into a device during assembling of the device.
14. Method of assembling a drug delivery device (100) or an electronic module (EM) for a drug delivery device (100), comprising:

providing a holding device (100) according to any one of the aspects 1 to 12 or the arrangement (1) according to aspect 13,

placing (240) the object (O) in the holding device (10),

wherein the object (O) is held within the holding device (10) when the first side (62) faces the at least one opening (21-1 to 21-4; 22-1 to 22-4), and

wherein the object (O) or another object (O) falls out of the holding device (10) when the second side (66) faces the at least one opening (21-1 to 21-4; 22-1 to 22-4),

placing (270) the object (O) into or onto an object receiving space within the drug delivery device (100) or within the electronic module (EM) using the holding device (10).
15. Drug delivery device (100), manufactured or manufacturable using the method of aspect 14, comprising:

a container receptacle, wherein the container receptacle is configured to receive a container (106) comprising a drug (Dr, M),

the container containing a drug (Dr, M), wherein the container (106) is arranged in the container receptacle, and

an electronic module (EM),

wherein the electronic module (EM) is electrically powered by an electrical power source comprised within or formed by the object (O), and

wherein a first surface (64) of the object (O) is arranged more proximally compared to a second surface (68) of the object (O),

wherein the first surface (64) is a flat surface that has a first maximal lateral extension or diameter,

wherein the second surface (68) is a flat surface that has a second maximal lateral extension or diameter,

wherein the first maximal lateral extension or diameter is greater than the second maximal lateral extension or diameter, and

wherein the first surface (64) has an opposite electrical polarity compared to the second surface (68).

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this application it means the possibility of doing so as well as the actual technical implementation. The present concepts of the present disclosure will be described with respect to preferred embodiments below in a more specific context namely drug delivery devices, especially drug delivery devices for humans or animals. The disclosed concepts may also be applied, however, to other situations and/or arrangements as well, e.g. for other injectors, spraying devices or inhalation devices. Alternatively, devices in other technical fields may be assembled using the proposed holding device and method(s), e.g. remote control, etc.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE FIGURES

For a more complete understanding of the presently disclosed concepts and the advantages thereof, reference is now made to the following description in conjunction with the accompanying drawings. The drawings are not drawn to scale. In the drawings the following is illustrated in:

FIG. 1 a plan view of a drug delivery device comprising a dosage knob as an example of a device of application for an object, the orientation of which is to be correctly defined,

FIG. 2 a sectional view of an embodiment of an electronic module/a dosage knob as an example of a device of application for a coin cell battery as an example of an object, the orientation of which is to be correctly defined, wherein the coin cell battery is comprised in the correct orientation,

FIG. 3 a sectional view of the electronic module/dosage knob of FIG. 2, wherein the coin cell battery is comprised in an incorrect orientation,

FIG. 4 a perspective view of an exemplifying embodiment of an assembly, the assembly comprising an embodiment of an object orientation-sensitive holding device and a coin cell battery as an exemplifying embodiment of an object, the orientation of which is to be correctly defined, wherein the coin cell is held in a receiving region of the device in the correct orientation,

FIG. 5 a perspective view of an exemplifying embodiment of an object orientation-sensitive holding device,

FIG. 6 a sectional view of an exemplifying embodiment of an assembly comprising an object orientation-sensitive holding device and a coin cell as an exemplifying embodiment of an object, wherein the coin cell is held and received in a receiving region of the device in the correct orientation,

FIG. 7 a sectional view of the assembly comprising the object orientation-sensitive holding device and the coin cell of FIG. 6, wherein the coin cell is received in the receiving region of the device in an incorrect orientation, and

FIG. 8 a flow diagram of a method of providing an object in a correct orientation thereof for use in a device of application of the object.

### DETAILED DESCRIPTION

Identical elements, elements of the same kind and identically or similarly acting elements may be provided with the same reference numerals in the drawings.

Reference may be made to a cylindrical coordinate system, i.e. each position may be defined by three coordinates: axial value (height, distance to zero plane), radial distance to axis and angle between current radial position and a plane that is defined as having angle zero. In this document the words “in an axial position” may mean having an axial coordinate.

The distal end D may be an end that is closer to a needle compared to a proximal end P.

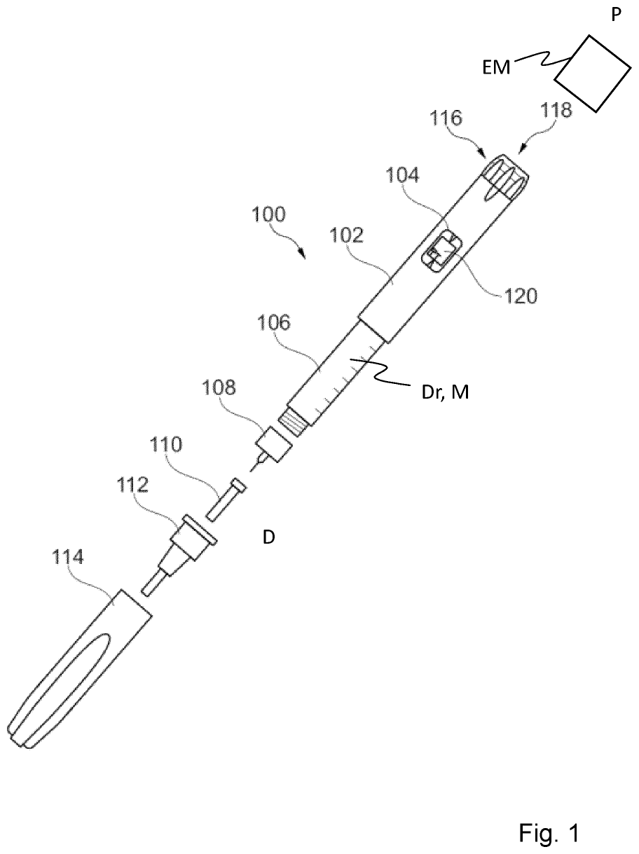

FIG. 1 illustrates a plan view of a drug delivery device 100 comprising a dosage knob 116 as an example of a device 90 of application for an object O, the orientation of which is to be correctly defined.

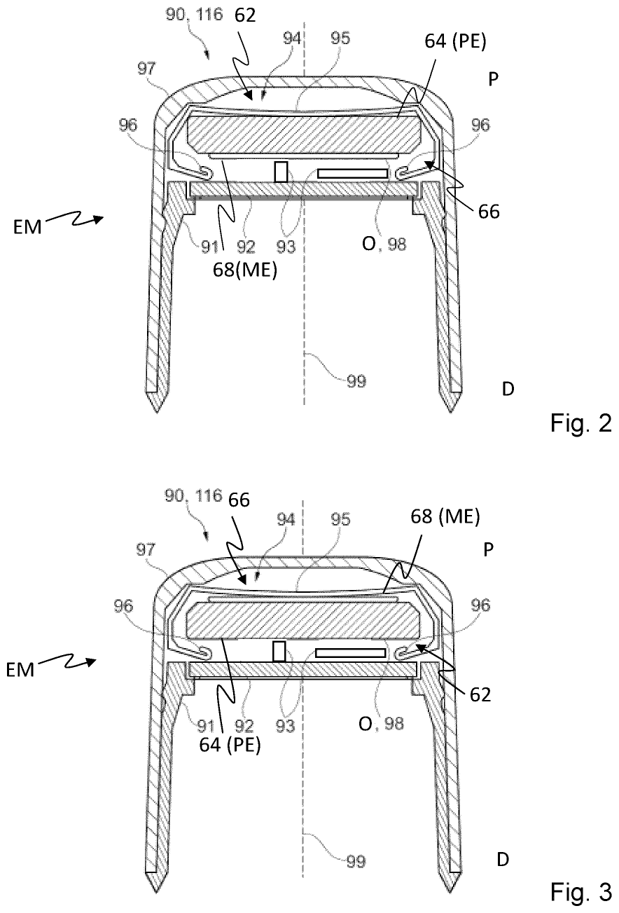

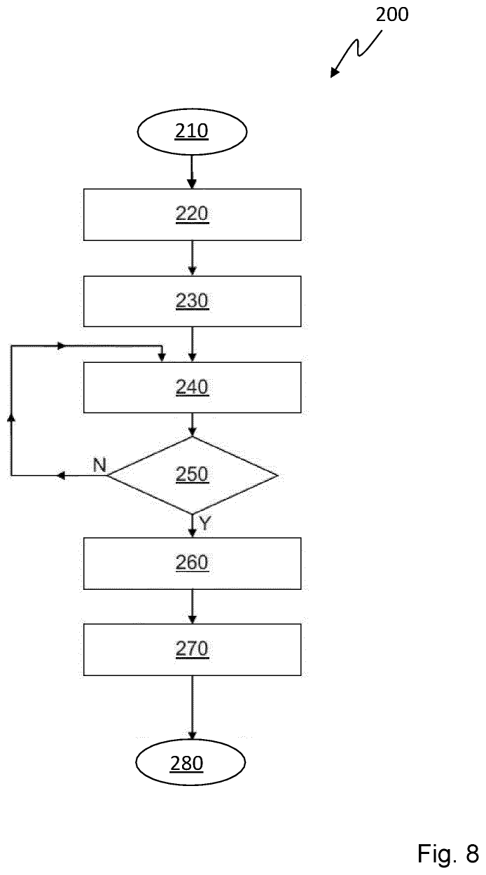

The drug delivery device 100, may be manufactured or may be manufacturable using the method mentioned below, see description of FIG. 8 and corresponding description. The drug delivery device 100 may comprise:
A housing 102,
A container receptacle, wherein the container receptacle may be configured to receive a container 106 comprising a drug Dr, M, see e.g. list of drugs as mentioned below.
Preferably, the container 106 containing a drug Dr, M, wherein the container 106 may be arranged in the container receptacle, and
An electronic module EM, preferably arranged within or on the housing 102, wherein the electronic module EM may be electrically powered by an electrical power source comprised within or formed by an object O (see FIGS. 2 to 7)

A first surface 64 (see e.g. FIG. 2) of the object O may be arranged more proximally P compared to a second surface 68 (see e.g. FIG. 2) of the object O. The first surface 64 may be a flat surface that has a first maximal lateral extension or diameter. The second surface 68 may be a flat surface that has a second maximal lateral extension or diameter. The first maximal lateral extension or diameter may be greater than the second maximal lateral extension or diameter, preferably by at least 1 percent or at least 2 percent of the second maximal lateral extension or diameter. The first surface 64 may have an opposite electrical polarity, e.g. plus potential, compared to the second surface 68 which may have e.g. minus electrical potential.

A window 104 may be used to display the amount of drug Dr, M selected by a user or patient that uses drug delivery device 100. Drug delivery device 100 may be a pen-type device of the axial extension type. A drive sleeve may extend proximally out of the housing 102 during dose selection or dose setting and may be pushed into the housing 102 during drug delivery performed manually by a user. However, drug delivery device 100 may also be another type of drug delivery device, e.g. an autoinjector, or a device comprising a torsion spring in order to supply a force to support drug delivery or to supply the whole force for drug delivery.

A needle 108 may be affixed to the container 106 or to the receptacle of the container 106. The container 106 may be a cartridge and the receptacle may be a cartridge holder. The needle 108 may have two sharp ends. One end of the needle 108 may be configured to be pierced through a sealing member of the cartridge. The other end of the needle 108 may be configured to pierced into the skin of the patient.

The needle 108 may be protected by an inner needle cap 110 and either an outer needle cap 112 or a cap 114 of the drug delivery device 100.

A drug Dr or medicament M dose, e.g. of insulin, to be ejected from drug delivery device 100 may be set e.g. by turning dosage knob 116, and a currently programmed or set dose may then be displayed via the dosage window 104, for instance in multiples of units. This may be achieved by an appropriate internal dose setting mechanism of drug delivery device 100. The indicia displayed in dosage window 104 may be provided on a dial or number sleeve 120.

The dosage knob 116 may also have an injection button 118, e.g. on its proximal end. Alternatively, the dosage knob 116 may be configured to act as an injection button 118. When the needle 108 is stuck into a skin portion of a patient, and then the dosage knob 116 or the injection button 118 is pushed in axial direction, e.g. distally, a drug (e.g. insulin) dose contained in the container 106 and the amount of which may be displayed in the dosage window 104 will be expelled from the drug delivery device 100.

The electronic module EM may be arranged within drug delivery device 100, e.g. as an integral component, e.g. within dosage knob 116. Alternatively, electronic module EM may be an additional part that may be coupled to the proximal end of drug delivery device 100, e.g. clipped, screwed, etc. onto dosage knob 116. The second alternative may allow usage of the electronic module EM on several drug delivery devices 100 of the same type, thereby providing multiple use of a comparably expensive electronic module EM. Moreover, only one power source (battery) or one set of power sources may be used a long time by a user or patient thereby reducing the environmental impact of the production and/or waste disposal of the electronic module EM.

An electrical source of energy may be provided to power the electrical module EM, e.g. an electrical battery. The electrical battery may be rechargeable or may be non-rechargeable. An example of a non-rechargeable battery is a battery in the shape of a coin cell battery 98, see e.g. FIGS. 2 and 3. An example of the coin cell is a coin cell of the type CR 1225. However, other types of coin cells may be used as well. According to international standard IEC (International Electrotechnical Commission) 60086-3, “C” stands for Lithium and “R” stands for a round form.

FIG. 2 illustrates a sectional view of an embodiment of an electronic module EM or of a dosage knob 116 as an example of a device of application 90 for a coin cell battery 98 as an example of an object O, the orientation of which is to be correctly defined, wherein the coin cell battery 98 is comprised in the correct orientation.

Object O is e.g. a coin cell 98. Coin cell 98 may comprise a first side 62 and a second side 66. First side 62 may comprise a first surface 64, e.g. an essentially flat surface. Second side 66 may comprise a second surface 68, e.g. an essentially flat surface. The first surface 64 may have a greater diameter and/or a greater surface area compared to the diameter and/or surface area of the second surface 68.

The first surface 64 may be part of a plus electrode PE of coin cell 98. Plus electrode PE may also extend circumferentially around the coin cell and may extend at the border of the second side 66. The second surface 68 may be part of a minus electrode ME of coin cell 98. An isolating region may be arranged between the plus electrode PE and the minus electrode ME at the second side 66, e.g. a sealing ring that may fulfill also a sealing function in order to seal the internal components (e.g. electrochemical components) of coin cell 98 from the outside of coin cell 98, e.g. from environment gas 30, humidity, dust, etc.

External or internal electronic module EM may comprise a base part 91 (e.g. a chassis). Base part 91 may be essentially cylindrical and may be configured to hold the major components of electronic module EM, e.g.:
A circuit board 92,
A coin cell 98,
Electro-mechanical contact elements that connect coin cell 98 and circuit board 92 electrically,
Sensor elements (not shown), and/or
Switching elements (not shown), etc.

Circuit board 92 may be e.g. a “printed” circuit board (PCB) carrying at least one layer of conductors that look like printed on the circuit board although photo lithographic techniques may usually be used to manufacture the circuit board 92.

Circuit board 92 may carry and connect several electronic components and/or electromechanical components in order to form a printed circuit board assembly (PCBA). Examples of components are e.g. a processor, a conductor, a resistor and/or capacitor(s), etc. Examples for sensor elements are radiation sensors, e.g. sensors for electromagnetic radiation, preferably within the visible spectrum or UV (ultraviolet) or IR (infrared) region of the spectrum.

The contact parts (elements) to contact coin cell 98 or other component may comprise at least one resilient sheet metal element, see e.g. positive electric contact clamp 94. A negative contact element may also comprise a resilient sheet metal element or another appropriate conducting surface. The negative contact element is not illustrated in FIGS. 2 and 3.

Exemplary, positive electric contact clamp 94 may comprise:
A positive electric contacting bridge 95, and
At least two positive electric contacting ends 96 connected to bridge 95, e.g. on opposite sides of bridge 95, and electrically conductive connected to circuit board 92.

Other shapes of positive contact clamp 94 are possible as well.

A cover part 97 may form a housing of the electronic module EM and may be arranged on base part 91 (e.g. chassis) as well. A dose dial interface may be arranged on the circumference of electronic module. An injection button interface may be formed on the proximal end face of electronic module EM.

A rotation axis 99 may be the axis, around which electronic module EM is rotated during dose selection (dose setting) and along which electronic module EM slides during dose injection.

The following example relates to dedicated assembling of a battery in the right orientation, e.g. to a coin cell assembly retainer with polarity rejection/detection. However, the concepts may be applicable to other objects O as well, especially to objects O with different surface configuration in edge region(s) and/or to flat objects.

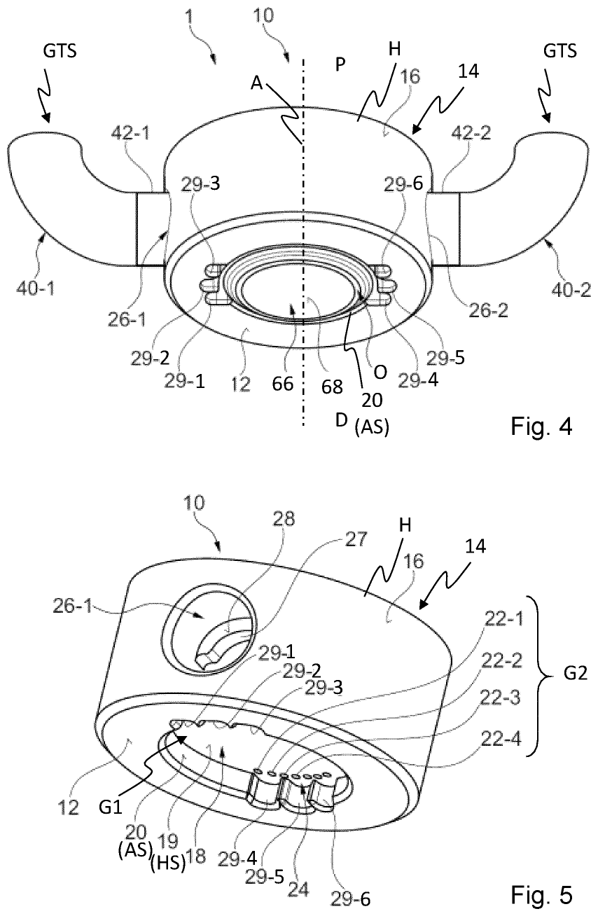

The embodiments relate to the use of gas suction applied to retain a coin cell 98 or another object O in an assembly tool, see holding device 10 (FIGS. 4 to 7) when it is correctly orientated but not when it is incorrectly orientated. Exemplary, the concepts are applicable to assembly tools for electronics devices which require a coin cell 98, and it may be intended to ensure the coin cell assembled with the correct polarity. This may be especially useful on a manual assembly line, where a human operator may be responsible for selecting the orientation of the coin cell 98 or another object O. The embodiments may be applicable to any component with an asymmetric geometry, e.g. relative to a mid-plane of the object O. Prototype assembling tools containing these features have performed well in early trials.

Thus, a manual assembly tool, see e.g. holding device 10 or similar devices may enable a coin cell 98 or other object O to be retained only when it is correctly orientated, thereby preventing incorrect assembly.

The right orientation of coin cell 98 may be the orientation depicted in FIG. 2, e.g. with the plus electrode PE arranged proximal of the minus electrode ME. Thus, the positive electric contact clamp 94 may contact the positive electrode PE on the first side 62 and/or the first surface 64. The minus electrode ME may be directed to the minus contact part that is not illustrated in FIGS. 2 and 3. The minus contact part/element may contact the second surface 68 on second sider 66 in order to provide the right electrical connection.

FIG. 3 illustrates a sectional view of the electronic module EM/dosage knob 116 of FIG. 2, wherein the coin cell battery O, 98 is comprised within the electronic module EM/dosage knob 116 in an incorrect orientation.

Thus, it may be possible to easily assemble a coin cell 98 to an add-on electronic module EM in two orientations. Each of the orientations connects the coin cell with a different polarity. If a coin cell is connected to the PCBA in the reverse polarity, the voltage will be applied to the circuit in the wrong direction and the electronic module EM will not function. It might also damage electrical components as they will have a supply voltage outside absolute maximum ratings, so the PCBA will need to be scrapped, e.g. since the damage will be invisible and will be difficult to detect and the assembly error cannot be corrected.

The incorrect orientation of coin cell 98 may be the orientation depicted in FIG. 3, e.g. with the minus electrode ME arranged proximal of the plus (positive) electrode PE. Thus, the positive electric contact clamp 94 may contact the minus (negative) electrode ME on the second side 66 and/or the second surface 68. The plus electrode PE may be directed to the minus contact part/element that is not illustrated in FIGS. 2 and 3. The minus contact part may contact the first surface 64 on first side 62 thereby providing a wrong electrical connection.

FIG. 4 illustrates a perspective view of an exemplifying embodiment of an assembly 1. The assembly 1 may comprise an embodiment of an object orientation-sensitive holding device 10 and the coin cell battery 98 as an exemplifying embodiment of an object O. The orientation of the coin cell battery 98 is correctly defined, wherein the coin cell 98 is held in a receiving region or within a retaining space 18, see FIG. 5, of the holding device 10 in the correct orientation. The coin cell 98 in the correct orientation is transferred to the electronic module in the correct orientation, thereby making sure that assembling of the coin cell 98 into electronic module takes also place in the correct orientation as is explained in more detail below, see FIG. 8 and corresponding description.

Holding device 10 may be used for holding an object O, e.g. a coin cell 98, during assembling of a medical device, e.g. of drug delivery device 100 or of electronic module EM that may form part of a medical device, e.g. of drug delivery device 100. Holding device 10 may comprise:
A holding structure HS (see FIG. 5), see e.g. abutting region 19 (abutting surface) in FIG. 5, and
An alignment structure AS, see e.g. lateral holding surface 20.

The holding structure HS may comprise at least one opening 21-1 to 21-4; 22-1 to 22-4, see FIG. 5.

The alignment structure AS may be configured to align an object O laterally, e.g. coin cell 98, relative to the holding structure HS. As already mentioned above, the object O may comprise a first side, e.g. 62, and a second side, e.g. 66, opposite to the first side, e.g. 62, of the object O. The first side, e.g. 62, may have a first surface configuration that is different to a second surface configuration of the second side e.g. 66. The at least one opening 21-1 to 21-4; 22-1 to 22-4 may be arranged to apply a negative pressure to the first side, e.g. 62, of the object O. The holding device 10 may be configured to hold the object O using the negative pressure only in a first orientation in which the first side, e.g. 62, of the object O faces and/or abuts the at least one opening 21-1 to 21-4; 22-1 to 22-4 but not in a second orientation in which the second side, e.g. 66, faces the at least one opening 21-1 to 21-4; 22-1 to 22-4.

A housing H of holding device 10 may comprise:
A first main surface 12, e.g. an essentially flat annular surface,
A second main surface 14, e.g. a circular flat surface,
A cylindrical outer surface 16,
A cylindrical retaining space 18, see FIG. 5,
An abutting region 19 (abutting surface), see FIG. 5, and
A lateral holding surface 20, e.g. a cylindrical wall.

An axis A may extend from a proximal end of housing H to a distal end of housing H. Housing H may have any appropriate shape, especially the outer surfaces may be modified, e.g. cuboid shaped.

Cylindrical retaining space 18, see FIG. 5, may be well adapted to the shape of object O, e.g. coin cell 98, e.g. it may have an essentially complementary shape. Thus, the same may be apply for abutting surface 19 (at least for one side of object O) and for lateral holding surface 20.

Gas flow recesses 29-1 to 29-3; 29-4 to 29-6 (air leakage recesses if air is used as gas) may be arranged adjacent to opening 21-1 to 21-4; 22-1 to 22-4, see e.g. FIG. 5.

A first gas hose 40-1, especially a first end 42-1 of gas hose 40-1, may be fluidically connected to openings 21-1 to 21-4. A second gas hose 40-2, especially a first end 42-2 of gas hose 40-2 may be fluidically connected to openings 22-1 to 22-4. The second end of first gas hose 40-1 and the second end of second gas hose 40-2 may be fluidically connected to a negative pressure source, e.g. a pump device. A Y-connector or other connector comprising at least three ports may be used to connect the second ends of gas hoses 40-1, 40-2 to the pressure source. Alternatively, two pressure sources may be used or a pressure source comprising at least two ports.

An electrical control unit may be connected to the pressure source in order to control pressure and/or switching on and switching off of the negative pressure source. Alternatively, a valve may be used to switch off or to switch on the negative pressure. The valve may be electrically connected to the control unit and fluidically to the first gas hose 40-1 and/or to the second gas hose 40-2. Alternatively, at least two valves may be used. Moreover, the control unit may have an input switch or may be connected to an input switch, e.g. operated by the foot of an operator, to switch off the negative pressure within holding device in order to release object O, e.g. coin cell 98, if transported to the final assembling place within a device, e.g. within drug delivery device 100 or within electronic module EM.

As depicted in FIG. 4, the second side 66 is the lower side and the second surface 68 is the lower surface. This is the correct orientation of object O, e.g. coin cell 98 as explained above with reference to FIG. 2.

The described concept may retain the coin cell 98 or another object O in the assembly tool (holding device 10) against the force of gravity using e.g. negative pressure gas flow creating suction. An external gas flow source, capable of producing a negative gas pressure, may be connected e.g. to the suction array, e.g. openings 21-1 to 21-4, using hoses, e.g. 40-1, 40-2, etc. The gas hoses, e.g. 40-1, 40-2, etc. may be assembled to the top tool (holding device 10) with an interference fit. The pipe offset surfaces 28, see FIG. 5 may prevent the hose openings from sitting flush against the end faces, which would constrict flow. The coin cell 98 or another object O may be aligned laterally by cylindrical retention faces. Gas leakage recesses 29-1 to 29-6 may ensure that the gas flow to the suction array is not restricted by radial contact between the coin cell 98 or another object O and top tool (holding device 10).

FIG. 5 illustrates a perspective view of an exemplifying embodiment of an object orientation-sensitive holding device. The alignment structure AS may comprise at least one alignment region provided to align the object O laterally, see e.g. cylindrical wall 20. The at least one alignment region may be provided (arranged) adjacent to the at least one opening 21-1 to 21-4; 22-1 to 22-4, preferably in order to hold the object O at the at least one edge region of the object O.

Cylindrical wall 20 may be essentially closed along its circumference, e.g. not regarding regions with gas flow regions 29-1 to 29-6. Alternatively, segments of a circular wall may be used, e.g. only two segments, e.g. combined with gas flow regions 29-1 to 29-6. Between the segments, there may be no lateral alignment region, e.g. there may be no wall or there may be another wall (segment) of a different shape, e.g. straight wall (segments).

Openings 21-1 to 21-4; 22-1 to 22-4, may be grouped in two groups G1, G2 separated from each other by a region that does not comprise openings. Openings within each group G1, G2 may be arranged in a one-dimensional array of openings. In the example, e.g. seven openings may be arranged along an arc, e.g. a circular arc. Of course, there may be less than seven or more than seven openings within on group G1, G2. The distance, especially the circumferential distance between adjacent openings 21-1 to 21-4; 22-1 to 22-4 of one group G1, G2 of openings 21-1 to 21-4; 22-1 to 22-4 may be constant, e.g. adjacent openings 21-1 to 21-4; 22-1 to 22-4 within one group G1, G2 may be arranged equidistantly. However, different distances between adjacent openings 21-1 to 21-4; 22-1 to 22-4 may be used, e.g. in order to generate same suction forces for each opening.

In the example, one group G1, G2 (array) of openings 21-1 to 21-4; 22-1 to 22-4 extends circumferentially only along an angle of less than 30 degrees or less than 20 degrees, e.g. more than 10 degrees.

Further, in the example, circular holes are used as openings 21-1 to 21-4; 22-1 to 22-4. However, alternatively, openings having or comprising other shapes may be used as well, e.g. comparably short slits or long slits, elliptical shape, etc.

Moreover, there may be other possibilities to arrange openings, e.g. without forming groups, along the whole circumference or into more than two groups G1, G2 along the whole circumference.

The holding structure HS may comprise at least one abutting region 19 configured to abut the second side 66 of the object O, 98 or alternatively the first side 62 and the second side 66. Abutting region 19 may extend in a plane, e.g. within a circular plane.

The at least one opening 21-1 to 21-4; 22-1 to 22-4 may be arranged at a different position compared to the at least one abutting region 19. This may result in a separation of the abutting function and of the suction function, especially in the case in which the second side 66 abuts the abutting region 19 and holding of object O does not take place.

Alternatively, the at least one opening 21-1 to 21-4; 22-1 to 22-4 may be arranged in the at least one abutting region 19. Thus, the abutting function and the suction function may be closely combined in abutting region 19.

There may also be two different abutting regions, depending on the side of the abject O that is directed to the abutting regions. Thus, a central abutting region 19 may be used for the case in which the second side 66 of object O, 98 faces the abutting region(s). Abutting regions around the openings 21-1 to 21-4; 22-1 to 22-4 may be used in the case that the first side 62 of the object O, 98 faces the abutting regions.

The different surface configuration(s) may be due to at least one of the following features:
a) A greater maximal lateral extension or diameter of a most prominent first surface 64 on the first side 62 compared to the maximal lateral extension or diameter of a most prominent second surface 68 on the second side 66. The first surface 64 may have a greater surface area compared to the second surface 68.
b) A smaller surface roughness of the first surface 64 on the first side 62 compared to the surface roughness on the second surface 68 on the second side 66. Thus, coin cell batteries may have a second side 68, e.g. minus electrode ME, that carries a pattern. The pattern may result in greater surface roughness compared to the first side 62 and its first surface 64, e.g. plus electrode PE. Thus, the first surface 64 may have a smoother surface profile compared to the second surface 68.
c) A chamfered circumferential region on the first side 62, wherein no such chamfered circumferential region may be present on the second side 66, preferably along the whole circumference. Thus, rotation of object O, 98 around its longitudinal axis does not matter, e.g. rotational alignment may not be necessary. This may be typically for batteries of coin cell shape type.
d) Other physical characteristics appropriate to interact with a gas stream (flow), e.g. an air stream (flow), through the at least one opening 21-1 to 21-4; 22-1 to 22-4 in order to achieve dedicated holding of the object O depending on its orientation relative to the holding device 10.

Holding device 10 may have two lateral openings 26-1, 26-2. An offset structure 27 may be arranged within a recess that extends radially inwards from lateral openings 26-1, 26-2. An offset surface 28 may be formed on offset structure 27. This will be explained below in more detail.

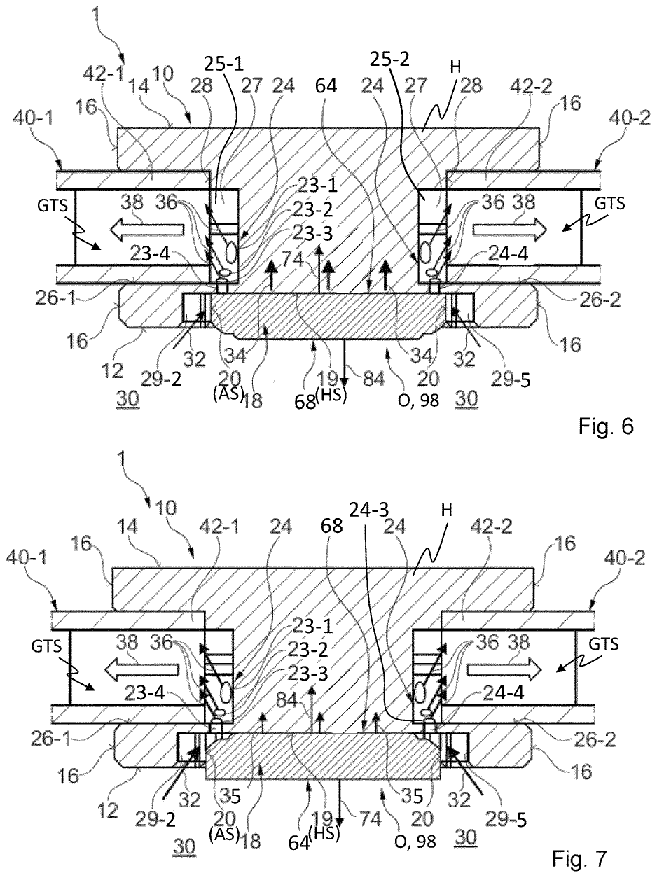

FIG. 6 illustrates a sectional view of an exemplifying embodiment of an assembly 1 comprising an object orientation-sensitive holding device 10 and a coin cell O, 98 as an exemplifying embodiment of an object O, wherein the coin cell 98 is held and received in a receiving region (e.g. cylindrical retaining space 18) of the holding device 10 in the correct orientation.

Holding device 10 may comprise at least one of the following features:
a) The at least one abutting region 19 may be arranged to provide an abutment surface for the object O in a first direction, preferably in an axial direction of the object O. The alignment structure AS may comprise at least one lateral holding surface 20 or surface portion that is configured to align the object O in a second direction that is different from the first direction, e.g. in the lateral direction, e.g. left and right in FIG. 6 or into the plane of FIG. 6 or out of the plane of FIG. 6, i.e. the plane of the paper sheet or of the display on which FIG. 6 is displayed.
b) The holding device 10 may comprise a gas transport structure GTS, e.g. an air transport structure. However, other gases may be used as well. The gas transport structure GTS may comprise at least one channel 23-1 to 23-4; 24-1 to 24-4; 25-1, 25-2 or tube 40-1, 40,2 connected to the at least one opening 21-1 to 21-4; 22-1 to 22-4 or connectable to the at least one opening 21-1 to 21-4; 22-1 to 22-4 and configured to be connected to a negative pressure supply source, e.g. a pump, preferably a diaphragm pump or a membrane pump.

Holding device 10 may comprise a housing H. Alternatively, a hose system and a holding plate may be used. The holding structure HS and/or the alignment structure AS may be provided on housing H or within housing H. Moreover, the housing H may comprise at least a part of the gas transport structure GTS, e.g. gas channels that are adjacent to the openings 21-1 to 21-4; 22-1 to 22-4, etc. The housing H may be made of plastic or of another appropriate material, e.g. of a metal.

Holding device 10 may comprise a cylindrical retaining space 18 that may be adapted to retain the object O and that may be delimited by the holding structure HS and/or by the alignment structure AS.

At least one first opening 21-1 to 21-4 of the at least one opening 21-1 to 21-4; 22-1 to 22-4 may be arranged on a first location. At least a one second opening 22-1 to 22-4 of the at least one opening 21-1 to 21-4; 22-1 to 22-4 may be arranged on a second location. The first location and the second location may be arranged on opposite lateral sides of the holding structure HS. At least one first opening 21-1 to 21-4 or all first openings 21-1 to 21-4 may be part of a first group G1 of the at least one opening 21-1 to 21-4; 22-1 to 22-4. The at least one second opening 22-1 to 22-4 or all second openings 22-1 to 22-4 may be part of a second group G2 of the at least one opening 21-1 to 21-4; 22-1 to 22-4.

The gas transport structure GTS may comprise at least one of the following:
a) At least one main channel 25-1, 25-2 that may be connected with at least a part of the at least one opening 21-1 to 21-4; 22-1 to 22-4,
b) A first main channel 25-1 that may be fluidically connected with at least a part of or all of the at least one opening 21-1 to 21-4 of the first group G1 and a second main channel 25-2 that may be different from the first main channel 25-1 and that may be fluidically connected with at least a part of or with all of the at least one opening 22-1 to 22-4 of the second group G2.
c) At least one gas flow recess 29-1 to 29-3; 29-4 to 29-6 adjacent e.g. near to the at least one opening 21-1 to 21-4; 22-1 to 22-4. The at least one gas flow (e.g. gas leakage) recess 29-1 to 29-3; 29-4 to 29-6 may be arranged such that the gas flow between the at least one gas flow recess 29-1 to 29-3; 29-4 to 29-6 and the at least one opening 21-1 to 21-4; 22-1 to 22-4 is restricted, e.g. essentially blocked by the object O when the first side 62 of the object O faces the at least one opening 21-1 to 21-4; 22-1 to 22-4 and is enabled when the second side 66 of the object O faces the at least one opening 21-1 to 21-4; 22-1 to 22-4 and/or the at least one abutting region 19.

The at least one gas flow recess 29-1 to 29-3; 29-4 to 29-6 may be configured such that at least two gas channels are formed between the at least one gas flow recess 29-1 to 29-3; 29-4 to 29-6 and at least two of the at least one opening 21-1 to 21-4; 22-1 to 22-4 when the second side 66 of the object O faces the at least one opening 21-1 to 21-4; 22-1 to 22-4. The at least two gas channels between the at least one gas flow recess 29-1 to 29-3; 29-4 to 29-6 and the at least two openings 21-1 to 21-4; 22-1 to 22-4 may be blocked or at least essentially blocked by the object O when the first side 62 faces the at least one opening 21-1 to 21-4; 22-1 to 22-4.

It goes without saying that more or less than three gas flow recess 29-1 to 29-3; 29-4 to 29-6 may be used on each lateral side of holding device 10.

Gas flow recess 29-1 to 29-3; 29-4 to 29-6 may have a cylindrical shape, e.g. with an open side to cylindrical wall 20. Alternatively, gas flow recess 29-1 to 29-3; 29-4 to 29-6 may have other shapes, e.g. elliptical bottom surface and elliptical cross sections parallel to bottom surface. Cuboid shapes may be used as well for gas flow recesses 29-1 to 29-3; 29-4 to 29-6.

Main channel 25-1, 25-2 may be fluidically connected to at least one of the openings 21-1 to 21-4; 22-1 to 22-4, preferably to the openings 21-1 to 21-4 of group G1 and to the openings 22-1 to 22-4 of group G2 respectively. Main channel 25-1, 25-2 may redirect the gas flow coming from the at least one opening 21-1 to 21-4; 22-1 to 22-4 (first flow direction 36) to a gas (e.g. air) flow second direction 38. An angle between the first direction 36 and the second direction 38 may be in the range of 80 degrees to 100 degrees, preferably about 90 degrees or 90 degrees.

The main channel 25-1, 25-2 may comprise at least one of the following:
a) An inlet portion comprising at least one inlet opening fluidically connected to a suction channel coming from the at least one opening 21-1 to 21-4; 22-1 to 22-4. The inlet portion may extend along the lower half of main channel 25-1, 25-2.
b) A tube or hose offset structure 27 arranged downstream to the inlet portion of the main channel 25-1, 25-2 and configured to define an offset between a sidewall of the main channel 25-1, 25-2 and a tube or hose 40-1, 40-2 fluidically connected to the main channel 25-1, 25-2, wherein preferably one end 42-1, 42-2 of the tube/hose 40-1, 40-2 may be arranged within the main channel 25-1, 25-2. A first end 42-1, 42-2 of tube or hose 40-1, 40-2 may abut against an offset surface 28 on offset structure 27 thereby defining a distance between tube or hose 40-1, 40-2 and the opposite sidewall of main channel 25-1, 25-2. This distance may prevent that channels 23-1 to 23-4, etc. are blocked by hose 40-1, e.g. by first end 42-1 of hose 40-1. A similar distance may prevent that channels 24-1 to 24-4, etc. are blocked by hose 40-2, e.g. by first end 42-2 of hose 40-2.
c) An outlet portion comprising an outlet opening, preferable an essentially circular outlet opening. Circular outlet opening may comprise two semi circles of different radius, e.g. a smaller one defined by offset structure 27 and a greater one defined by the inlet portion.

There may be the following channels:
Suction channels 23-1 to 23-4, etc. between inlet portion of main channel 25-1 and respective openings 21-1 to 21-4 etc., and
Suction channels 24-1 to 24-4, etc. between inlet portion of main channel 25-1 and respective openings 22-1 to 22-4.

Negative pressure supply openings 26-1, 26-2 may be fluidically connected with a negative pressure source (not shown) via hose 40-1 and 40-2 respectively. Rigid pipes may be used instead of flexible hoses 40-1 and 40-2. The negative pressure source may be e.g. a pump.

As illustrated in FIG. 6, the following comparably weak gas flows (e.g. if compared with corresponding flows as illustrated in FIG. 7) may be established between a gas (e.g. air) environment 30 and the negative pressure source:
Gas flows 32 from environment 30 through gas flow recess(es) 29-2, 29-5, etc., through a small gap between first surface 64 and openings 21-1 to 21-4, etc.; 22-1 to 22-4, etc. to openings 21-1 to 21-4, etc., 22-1 to 22-4, etc. This means that the object O, e.g. a battery in the shape of a coin cell 98, may block the resulting gas stream essentially or completely, e.g. the flow may be less than 30 percent or less than 10 percent of the corresponding flow relevant in FIG. 7.
Gas flows 36 from openings 21-1 to 21-4, etc., 22-1 to 22-4, etc. through suction channels 23-1 to 23-4, etc.; 24-1 to 24-4, etc. into inlet portion of main channel 25-1, 25-2 respectively.
Gas flow 38 from outlet portion of main channel 25-1, 25-2 respectively into hose 40-1, 40-2.
Further gas flows (not illustrated) through hose 40-1, 40-2, optional connections and/or valves to negative pressure source (also not shown).

A resulting suction force 34 may be comparably high, e.g. if compared with suction force 35 relevant for the embodiment and arrangement shown in FIG. 7 and described in detail below. Thus, object O (e.g. coin cell 98) may be hold with a comparably high holding force in the orientation as illustrated in FIG. 6, e.g. a first normal vector 74 of the first surface 64 (e.g. plus electrode PE) is directed (points) to holding device 10. A second normal vector 84 of the second surface 68 (e.g. minus electrode ME) is directed (points) away from holding device 10. Holding of object O, e.g. of coin cell 98 may be possible since the holding force is higher than the gravity force exerted to the object O, 98.

A holding system may comprise at least one of:
A holding device 10 according to any one of the embodiments mentioned above,
A negative pressure source, and
A connection system connecting or configured to connect the holding device 10 and the negative pressure source.

An arrangement 1 may comprise the holding device 10 according to any one of the embodiments mentioned above or the holding system as mentioned above and an object O, 98 configured to be held in the holding system in order to place the object O, 98 into a device, e.g. a drug delivery device 100 or electronic module EM, during assembling of the device, e.g. of device 100, or of the electronic module EM.

When the operator places a correctly orientated coin cell 98 or other object O in the top tool 10 it will cover the suction array, e.g. openings 21-1 to 22-4, etc., which may be positioned towards the edge of the recess, e.g. retaining space 18, restricting gas flow. The resulting suction force 34 is more than the gravitational pull, so the coin cell 98 or other object O is held in the top tool (holding device 10).

FIG. 7 illustrates a sectional view of the assembly 1 comprising the object orientation-sensitive holding device 10 and the coin cell 98 of FIG. 6, wherein the coin cell 98 or another object is received in the receiving region, e.g. retaining space 18, of the holding device 10 in an incorrect orientation.

If the operator places an incorrectly orientated coin cell 98 or other object O in the top tool, e.g. holding device 10, it will not cover the suction array, e.g. openings 21-1 to 22-4, etc., due to e.g. the reduced diameter region on its negative polarity side (e.g. minus electrode ME) so that there is no suction or only a very weak suction applied to the coin cell 98 or other object O and it falls out of the tool, e.g. holding device 10, under the force of gravity As illustrated in FIG. 7, the following comparably strong gas flows (e.g. if compared with corresponding gas flows as illustrated in FIG. 6) may be established between a gas (e.g. an air) environment 30 and the negative pressure source:
Gas flows 32 from environment 30 through gas flow recess(es) 29-2, 29-5, etc., through a channel formed between holding device 10 and the border or object O, coin cell 98, e.g. there may be a comparably great gap between edge of object O on the second side 66 and openings 21-1 to 21-4, etc.; 22-1 to 22-4, etc. to openings 21-1 to 21-4, etc., 22-1 to 22-4, etc. This means that the object O, e.g. battery in the shape of a coin cell 98, may allow a gas (e.g. air) stream or flow 32, especially a gas flow 32 that is much higher than gas flow 32 relevant for the arrangement illustrated in FIG. 6.
Gas flows 36 from openings 21-1 to 21-4, etc., 22-1 to 22-4, etc. through suction channels 23-1 to 23-4, etc.; 24-1 to 24-4, etc. into inlet portion of main channel 25-1, 25-2 respectively. Gas flow 36 illustrated in FIG. 7 may be much higher compared to gas flow 36 as illustrated in FIG. 6, see bolder arrows used in FIG. 7.
Gas flow 38 from outlet portion of main channel 25-1, 25-2 respectively into hose 40-1, 40-2.
Further gas flows (not illustrated) through hose 40-1, 40-2, optional connections and/or valves to negative pressure source (also not shown).

A resulting suction force 35 may be comparably low, e.g. if compared with suction force 34 relevant for the embodiment and arrangement shown in FIG. 6 and described in detail above, see thinner arrows used in FIG. 7 to visualize suction force 35. Thus, object O (e.g. coin cell 98) is not hold or is only hold with a comparably low holding force (e.g. lower than the force due to gravity or exerted by gravity to object O/coin cell 98) in the orientation as illustrated in FIG. 7, e.g. a first normal vector 74 of the first surface 64 (e.g. plus electrode PE) is directed (points) away from holding device 10. A second normal vector 84 of the second surface 68 (e.g. minus electrode ME) is directed (points) to holding device 10. Thus, transport of object O, e.g. coin cell 98, to an assembly location is not possible, e.g. to an end of a drug delivery device 100 or to another device of application, e.g. electronic module EM or dosage knob 116.

FIG. 8 illustrates a flow diagram of a method 200 of providing an object O, e.g. a coin cell 98, in a correct orientation thereof for use in a device of application 90 of the object O see FIGS. 2 and 3. The method of assembling of a drug delivery device 100 or of an electronic module EM for a drug delivery device 100 may comprise:
Using or providing a holding device 10 according to any one of the embodiments mentioned above or the holding system as mentioned above,
Placing 240 the object O, 98 in the holding device 10,

wherein the object O may be held within the holding device 10 when the first side 62 faces the at least one opening 21-1 to 21-4; 22-1 to 22-4, e.g. right orientation, and

wherein the object O or another object O may fall out of the holding device 10 when the second side 66 faces the at least one opening 21-1 to 21-4; 22-1 to 22-4, e.g. wrong orientation.
Preferably transporting or transferring 260 the object O, 98 to an assembling position. Alternatively, other components to be assembled may be transported or transported to a position below holding device 10. Object O, 98 will only be hold in the meantime within holding device 10 if it is placed therein in the right orientation, see e.g. FIG. 6. Otherwise it will fall out of holding device 10 in the meantime, i.e. if placed into holding device 10 in the wrong orientation, see e.g. FIG. 7.
Placing 270 the object O, 98 into or onto an object receiving space within the drug delivery device 100 or within the electronic module EM using the holding device 10, e.g. releasing it by switching off negative gas (e.g. air) pressure or by reducing the absolute value of the negative gas (air) pressure.

In more detail, the method 200 may begin at a step 210. In step 220, there may be provided an object orientation-sensitive holding device 10 for selectively holding an object O, 98 according to an orientation 74, 84 of the object O, 98 relative to the holding device 10. For example, the object orientation-sensitive holding device 10 may be the holding device 10 shown FIGS. 4 through 7.

In step 230, the holding device 10 may be set in operation. The object orientation-sensitive holding device 10 may comprise a retaining space 18 (receiving region) adapted to receive the object O, 98 in a first orientation 74 relative to the holding device 10 or in a second orientation 84 relative to the holding device 10. Herein, the holding device 10 may be adapted to selectively hold the object O, 98 in the retaining space 18 if in operation of the holding device 10 the object O, 98 is received in the retaining space 18 in a first orientation 74 of the object O, 98 (as shown in FIG. 6). On the contrary, the holding device 10 may be adapted to not hold the object O, 98 in the retaining space 18 if in operation of the holding device 10 the object O, 98 is received in the retaining space 18 in a second orientation 84 of the object O, 98 (as shown in FIG. 7). A negative pressure source may be switched on or negative pressure may be applied by controlling respective valve(s) of the holding system as mentioned above.

In step 240, a new object O, 98 may be placed in the retaining space 18 (receiving region) of the holding device 10, wherein the object O, 98 is in one of a first orientation 74 and a second orientation 84 relative to the holding device 10. For example, the object O, 98 may be a coin cell battery O, 98 and the device 90 of application may be a dosage knob 116, as shown in FIGS. 2 and 3 or an electronic module EM.

If the object O, 98 is not held in and falls out of the retaining space 18 (receiving region), that is at step 250, N, the flow of the method may return to step 240. This may be regarded as an implicit check whether the object O, 98 is arranged in the right orientation within holding device 10.

Step 240 is performed with the same object O, 98 in the other orientation or with a new object O, 98 in the right orientation, e.g. the same object O, 98 or a new object O, 98 is placed in the retaining space 18.

If the object O, 98 is held in the retaining space 18, that is at step 250, Y, the flow of the method may continue to step 260, where the object O, 98 may be transferred from the retaining space 18 of the object orientation-sensitive holding device 10 to a new device 90 of application of the object O, 98, e.g. electronic module EM. As mentioned already above, alternatively or additionally, the device to be assembled may also be transferred or transported to a position below the holding device 10.

In step 270, negative gas (air) pressure may be switched off, e.g. to release object O, 98 from holding device 10. Object O, 98 may be directly placed into the device/module to be assembled, e.g. in its final assembling place and assembling position. Further, objects O, 98 may be assembled in the same way. The method 200 may end at a step 280.

The method 200 may comprise at least one intermediate step that has not been described. Moreover, other modifications of method 200 are possible as well.

### List of Drugs

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY320176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein.

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. The embodiments mentioned in the first part of the description may be combined with each other. The embodiments of the description of figures may also be combined with each other. Further, it is possible to combine embodiments mentioned in the first part of the description with examples of the second part of the description which relates to FIGS. 1 to 8.

### REFERENCE NUMERALS

1 arrangement
10 holding device
12 first main surface
14 second main surface
16 cylindrical outer surface
18 cylindrical retaining space
19 abutting region (surface)
20 lateral holding surface (cylindrical wall)
21-1 to 21-4, etc. suction opening
22-1 to 22-4, etc. suction opening
G1, G2 group
23-1 to 23-4 suction channel
24-1 to 24-4 suction channel
25-1, 25-2 main channel
26-1, 26-2 negative pressure supply opening
27 offset structure
28 offset surface
29-1 to 29-3 gas flow recess (air)
29-4 to 29-6 gas flow recess (air)
30 gas environment
32 gas flow
34, 35 suction force
36, 38 gas flow
40-1, 40-2 gas hose
42-1, 42-2 first end
O object (e.g. coin cell)
62 first side
64 first surface
66 second side
68 second surface
PE plus electrode
ME minus electrode
EM electronic module
Dr, M drug, medicament
P proximal
D distal
A axis
H housing
AS alignment structure
HS holding structure
GTS gas transport structure
74 first normal vector
84 second normal vector
90 device of application/component
91 base part
92 circuit board
93 negative electric contact part (and/or electronic component, capacitor etc.)
94 positive electric contact clamp
95 positive electric contacting bridge
96 positive electric contacting end
97 cover part
98 electric component/coin cell battery/object
99 rotation axis
100 injection device
102 housing
104 window
106 container
108 needle
110 inner needle cap
112 outer needle cap
114 other cap
116 dosage knob
118 injection button
120 dial or number sleeve
200 method of providing a correct orientation of an object
210 BEGIN
220 providing an object orientation-sensitive holding device
230 setting the device in operation
240 placing a new object in the receiving region of the object orientation-sensitive holding device
250 checking whether the object is held in the receiving region
260 transferring the object from the receiving region to a new device of application
270 switch off negative gas pressure
280 END

1.-15. (canceled)

16. A holding device for holding an object during assembly of a medical device, the holding device comprising:
a holding structure; and
an alignment structure,
wherein the holding structure comprises at least one opening,
wherein the alignment structure is configured to align the object relative to the holding structure,
wherein the object is a coin cell battery,
wherein the object comprises a first side and a second side opposite to the first side of the object,
wherein the first side has a first surface configuration that is different to a second surface configuration of the second side,
wherein the at least one opening is arranged to apply a negative pressure to the first side of the object, and
wherein the holding device is configured to hold the object using the negative pressure only in a first orientation in which the first side of the object faces the at least one opening but not in a second orientation in which the second side faces the at least one opening.

17. The holding device according to claim 16, wherein the alignment structure comprises at least one alignment region configured to align the object laterally, and
wherein the at least one alignment region is adjacent to the at least one opening and configured to hold the object at at least one edge region of the object.

18. The holding device according to claim 16, wherein the holding structure comprises at least one abutting region configured to abut (i) the second side of the object or (ii) the first side and the second side,
wherein the at least one opening is arranged at a different position compared to the at least one abutting region or wherein the at least one opening is arranged in the at least one abutting region.

19. The holding device according to claim 16, wherein the different surface configurations are due to at least one of the following features:
a greater maximal lateral extension or diameter of a most prominent first surface on the first side compared to the maximal lateral extension or diameter of a most prominent second surface on the second side,
a smaller surface roughness of a first surface on the first side compared to the surface roughness on a second surface on the second side,
a chamfered or rounded circumferential region on the first side, wherein no such circumferential region is present on the second side, or
other physical characteristics appropriate to interact with a gas stream through the at least one opening in order to achieve dedicated holding of the object depending on its orientation relative to the holding device.

20. The holding device according to claim 16, comprising at least one of the following features:
a) wherein at least one abutting region is arranged to provide an abutment surface for the object in a first direction, and
wherein the alignment structure comprises at least one lateral holding surface or surface portion that is configured to align the object in a second direction that is different from the first direction, or
b) wherein the holding device comprises a gas transport structure,
wherein the gas transport structure comprises at least one channel or tube connected to the at least one opening or connectable to the at least one opening and configured to be connected to a negative pressure supply source.

21. The holding device according to claim 16, further comprising a housing,
wherein the holding structure and the alignment structure are arranged on the housing or within the housing, and
wherein the housing comprises at least a part of a gas transport structure.

22. The holding device according to claim 16, further comprising a cylindrical retaining space, wherein the cylindrical retaining space is adapted to retain the object and is delimited by the holding structure and by the alignment structure.

23. The holding device according to claim 16,
wherein the holding structure comprises a plurality of openings, wherein at least one first opening of the plurality of openings is arranged on a first location and wherein at least one second opening of the plurality of openings is arranged on a second location, and wherein the first location and the second location are arranged on opposite lateral sides of the holding structure, and/or
wherein the holding device is configured such that the object is held by the negative pressure applied by the at least one opening in an edge region of the object in the first orientation but not at a center region.

24. The holding device according to claim 21, wherein the housing comprises at least a part of a gas transport structure that comprises at least one of the following:
a) at least one main channel connected with at least a part of the at least one opening,
b) a first main channel fluidically connected with at least a part of the at least one opening of a first group, and a second main channel different from the first main channel and fluidically connected with at least a part of the at least one opening of a second group, or
c) at least one gas flow recess adjacent to the at least one opening, wherein the at least one gas flow recess is arranged such that gas flow between the at least one gas flow recess and the at least one opening is restricted essentially by the object when the first side of the object faces the at least one opening and is enabled when the second side of the object faces at least one of the at least one opening and the at least one holding region.

25. The holding device according to claim 21, wherein the holding structure comprises a plurality of openings,
wherein the housing comprises at least a part of a gas transport structure that comprises comprises at least one gas flow recess configured such that at least two gas channels are formed between the at least one gas flow recess and at least two of the plurality of openings when the second side faces at least one of the plurality of openings, and
wherein the at least two gas channels between the at least one gas flow recess and the at least two openings are blocked or at least essentially blocked by the object when the first side faces at least one of the plurality of openings.

26. The holding device according to claim 16, further comprising at least one main channel that is fluidically connected to the at least one opening and that redirects gas flow from a first flow direction at the at least one opening to a second direction, wherein an angle between the first flow direction and the second direction is in a range of 80 degrees to 100 degrees, and wherein the main channel comprises at least one of the following:
a) an inlet portion comprising at least one inlet opening fluidically connected to a suction channel coming from the at least one opening,
b) a tube or hose offset structure arranged downstream to an inlet portion of the main channel and configured to define an offset between a sidewall of the main channel and a tube fluidically connected to the main channel, or
c) an outlet portion comprising an outlet opening.

27. A holding system comprising:
a holding device for holding an object, the holding device comprising:
a holding structure; and
an alignment structure,
wherein the holding structure comprises at least one opening,
wherein the alignment structure is configured to align the object relative to the holding structure,
wherein the object is a coin cell battery,
wherein the object comprises a first side and a second side opposite to the first side of the object, wherein the first side has a first surface configuration that is different to a second surface configuration of the second side;
a negative pressure source; and
a connections system connecting or configured to connect the holding device and the negative pressure source,
wherein the at least one opening is arranged to apply the negative pressure to the first side of the object, and
wherein the holding device is configured to hold the object using the negative pressure only in a first orientation in which the first side of the object faces the at least one opening but not in a second orientation in which the second side faces the at least one opening.

28. The holding system according to claim 27, wherein the alignment structure comprises at least one alignment region configured to align the object laterally, and
wherein the at least one alignment region is adjacent to the at least one opening and configured to hold the object at at least one edge region of the object.

29. The holding system according to claim 27, wherein the holding structure comprises at least one abutting region configured to abut (i) the second side of the object or (ii) the first side and the second side,
wherein the at least one opening is arranged at a different position compared to the at least one abutting region or wherein the at least one opening is arranged in the at least one abutting region.

30. The holding system according to claim 27, wherein the different surface configurations are due to at least one of the following features:
a greater maximal lateral extension or diameter of a most prominent first surface on the first side compared to the maximal lateral extension or diameter of a most prominent second surface on the second side,
a smaller surface roughness of a first surface on the first side compared to the surface roughness on a second surface on the second side,
a chamfered or rounded circumferential region on the first side, wherein no such circumferential region is present on the second side, or
other physical characteristics appropriate to interact with a gas stream through the at least one opening in order to achieve dedicated holding of the object depending on its orientation relative to the holding device.

31. The holding system according to claim 27, comprising at least one of the following features:
a) wherein at least one abutting region is arranged to provide an abutment surface for the object in a first direction, and
wherein the alignment structure comprises at least one lateral holding surface or surface portion that is configured to align the object in a second direction that is different from the first direction, or
b) wherein the holding device comprises a gas transport structure,
wherein the gas transport structure comprises at least one channel or tube connected to the at least one opening or connectable to the at least one opening and configured to be connected to a negative pressure supply source.

32. The holding system according to claim 27, further comprising a housing,
wherein the holding structure and the alignment structure are arranged on the housing or within the housing, and
wherein the housing comprises at least a part of a gas transport structure.

33. The holding system according to claim 27, further comprising a cylindrical retaining space, wherein the cylindrical retaining space is adapted to retain the object and is delimited by the holding structure and by the alignment structure.

34. The holding system according to claim 27,
wherein the holding structure comprises a plurality of openings, wherein at least one first opening of the plurality of openings is arranged on a first location and wherein at least one second opening of the plurality of openings is arranged on a second location, and wherein the first location and the second location are arranged on opposite lateral sides of the holding structure, and/or
wherein the holding device is configured such that the object is held by the negative pressure applied by the at least one opening in an edge region of the object in the first orientation but not at a center region.

35. A method of assembling a drug delivery device or an electronic module for a drug delivery device, comprising:
providing a holding device for holding an object during assembly of the drug delivery device or an electronic module for a drug delivery device, the holding device comprising:
a holding structure; and
an alignment structure,
wherein the holding structure comprises at least one opening,
wherein the alignment structure is configured to align the object relative to the holding structure,
wherein the object is a coin cell battery,
wherein the object comprises a first side and a second side opposite to the first side of the object,
wherein the first side has a first surface configuration that is different to a second surface configuration of the second side,
wherein the at least one opening is arranged to apply a negative pressure to the first side of the object, and
wherein the holding device is configured to hold the object using the negative pressure only in a first orientation in which the first side of the object faces the at least one opening but not in a second orientation in which the second side faces the at least one opening;
placing the object in the holding device,
wherein the object is held within the holding device when the first side faces the at least one opening, and
wherein the object or another object falls out of the holding device when the second side faces the at least one opening; and
placing the object into or onto an object receiving space within the drug delivery device or within the electronic module using the holding device.
